(19) United States
(12) Patent Application Publication
(10) Pub. No.: US 2011/0053951 A1
(43) Pub. Date: Mar. 3, 2011

(54) METHODS FOR TREATING DISORDERS USING NMDA NR2B-SUBTYPE SELECTIVE ANTAGONIST

(75) Inventors: John Alan Kemp, Basel (CH); Timothy Tasker, Isleworth (GB)

(73) Assignee: EVOTEC NEUROSCIENCES GMBH, Hamburg (DE)

(21) Appl. No.: 12/919,804

(22) PCT Filed: Mar. 26, 2009
(86) PCT No.: PCT/EP09/02241

§ 371 Date: Aug. 27, 2010

Related U.S. Application Data
(60) Provisional application No. 61/040,087, filed on Mar. 27, 2008.

(51) Int. Cl. A61K 31/501; A61P 25/28; A61P 25/04; A61P 25/24; A61P 25/30; A61P 25/00; A61P 25/16

(52) U.S. Cl. 514/252.05

(57) ABSTRACT

A method of treating, preventing or ameliorating a disease or condition by inhibiting NR2B subunit containing NMDA receptors using a compound according to formula (I) or a pharmaceutically acceptable salt thereof:

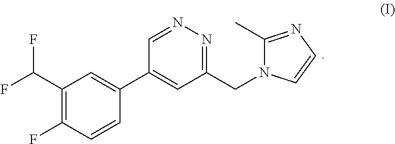

### BACKGROUND OF THE INVENTION

Extensive studies over the past twenty years have indicated that NMDA receptors play an important role in Alzheimer's disease (AD), Parkinson's disease, and pain sensation. The clinical development of non-selective NMDA receptor antagonists in general has, however, been limited by unfavorable side-effects, such as hallucinations.

In the early 1990s, it was found that multiple NMDA receptor subtypes exist, which contain different NR2(A-D) subunits. NR2B subunit containing receptors have been implicated in modulating functions, such as learning, memory processing, attention, emotion, mood, and pain perception, as well as being involved in a number of human disorders.

Compounds selectively targeting the NR2B subunit containing NMDA receptors are generally known. For example, U.S. Pat. No. 7,005,432 discloses a broad variety of substituted imidazol-pyridazine derivatives that are NMDA receptor subtype selective blockers, which are said to be useful in the therapy of CNS disorders. It discloses that the dosage can vary within wide limits. In the case of oral administration, the dosage is in the range of about 0.1 mg per dosage to about 1000 mg per day of a compound of general formula I of U.S. Pat. No. 7,005,432, although the upper limit can also be exceeded when this is shown to be indicated.

Due to various factors, such as gastro-intestinal absorption, plasma-protein binding, and the ability of compounds to pass the blood-brain-barrier, it is not possible to predict in what amount a specific imidazol-pyridazine derivative would be effective.

### SUMMARY OF THE INVENTION

The present invention provides a method for treating, preventing, or ameliorating a disease or condition by inhibiting NR2B subunit containing NMDA receptors. NR2B subunit containing receptors have been implicated in modulating functions, such as learning, memory processing, attention, emotion, mood, and pain perception, as well as being involved in a number of human disorders. Such disorders include, for example, cognitive impairment, neurodegenerative disorders, such as Alzheimer's disease and Parkinson's disease, pain (e.g., chronic or acute pain; neuropathic pain; post-operative pain), depression, attention deficit hyperactivity disorder, and addiction.

This method involves the use of 5-(3-difluoromethyl-4-fluoro-phenyl)-3-(2-methyl-imidazol-1-yl-methyl)-pyridazine, which is represented by formula (I) below:

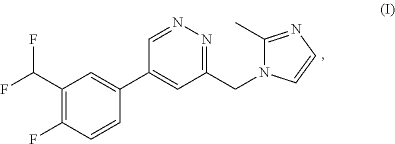

or a pharmaceutically acceptable salt thereof.

Preferably, the amount of the compound of formula (I) or its pharmaceutically acceptable salt that is administered for the treatment is from about 2 mg to about 50 mg per day. The total daily dose may be administered as single or divided doses. Such daily treatment amount or total daily dose may be from about 5 mg to about 45 mg, from about 6 mg to about 35 mg, from about 8 mg to about 30 mg, from about 10 mg to about 25 mg, from about 12 mg to about 20 mg, from about 14 mg to about 18 mg, from about 15 mg to about 18 mg or any range among all of the above-listed amounts. For example, the daily treatment amount is from about 2 mg, about 5 mg, about 6 mg, about 8 mg or about 10 mg to about 12 mg, about 14 mg, about 15 mg, about 16 mg, about 18 mg, about 20 mg, about 25 mg, about 30 mg, or about 35 mg. In particular, the daily treatment amount is from about 2 mg, or about 4 mg to about 20 mg, about 25 mg, or about 30 mg.

The compound of formula (I) or its pharmaceutically acceptable salt may be administered orally in a form of a pharmaceutical composition comprising the compound of formula (I) or its pharmaceutically acceptable salt in the desired amount and the appropriate carrier.

The subjects to be treated in accordance with the present invention are humans.

The phrase “pharmaceutically acceptable” is employed herein to refer to those compounds, materials, compositions, and/or dosage forms, which are, within the scope of sound medical judgment, suitable for use in contact with the tissues of human beings without excessive toxicity, irritation, allergic response, or other problem or complication, commensurate with a reasonable benefit/risk ratio.

As used herein, “pharmaceutically acceptable salts” refer to derivatives of the disclosed compound wherein the parent compound is modified by making salts thereof. Pharmaceutically acceptable salts, in particular acid addition salts, can be manufactured according to methods, which are known per se and familiar to any person skilled in the art. Lists of suitable salts are found in Remington's Pharmaceutical Sciences, 17th ed., Mack Publishing Company, Easton, Pa., 1985, p. 1418, the disclosure of which is hereby incorporated by reference.

As used herein, the “total daily dose” or “daily treatment amount” refers to the total amount of the compound of formula (I) and/or its pharmaceutically acceptable salt to be administered within a 24-hour period. It should be understood that treatment by administration of the total daily dose or daily treatment amount in accordance with the present invention does not require that this administration occur on a daily basis, but only that the amount administered in a 24-hour period be within the total daily dose range. For example, the drug may be administered daily, every other day, or at other intervals.

### BRIEF DESCRIPTION OF THE DRAWINGS

FIG. 1. shows plasma concentrations and cerebrospinal fluid (CSF) concentrations of the compound of formula (I) obtained from plasma or CSF samples from six individual subjects. Subjects were treated with daily dosages of 8 mg of the bis HCl salt of the compound of formula (I) for 8 consecutive days and samples were taken as indicated at times after dosing on day 8.

FIG. 2. shows representative slices of continuous arterial spin labeling (CASL) perfusion images from a single subject.

FIG. 3 is a chart showing global regional cerebral blood flow (rCBF) for the whole brain region acquired in the continuous arterial spin labeling images. Bars on the left, middle, and right correspond to placebo, 8 mg, and 15 mg of administered bis HCl salt of compound of formula (I), respectively.

FIG. 4 shows how administration of the bis HCl salt of compound of formula (I) increases rCBF in the anterior cingulate shown on (a) ‘glass brain’ projections thresholded for statistical significance for clusters across the whole brain at p<0.05 (voxel threshold p<0.001); (b) overlayed significance maps on the single subject T1 template image provided with Statistical Parametric Mapping software (SPM v5.0); and (c) cluster values extracted and plotted as means and standard errors of the means. Bars on the left, middle, and right of FIG. 4 (c) correspond to placebo, 8 mg, and 15 mg of administered bis HCl salt of compound of formula (I), respectively.

FIG. 5 shows glass brain maximum intensity projections showing the statistically significant clusters of activation during retrieval in the Paired Associates Learning task (PAL) after administration of placebo (left: upper and lower panel), 8 mg of bis HCl salt of compound of formula (I) (middle: upper and lower panel), and 15 mg of bis HCl salt of compound of formula (I) (right: upper and lower panel), respectively. Upper images are from the left and lower images are from above.

FIG. 6 shows dose response curves for the activation contrast in respective brain regions as indicated. Points on the left, middle, and right correspond to placebo, 8 mg and 15 mg of administered bis HCl salt of compound of formula (I), respectively. Data are extracted from activation patterns during retrieval in the Paired Associates Learning task (PAL). VLPFC in FIG. 6 refers to ventrolateral prefrontal cortex.

### DETAILED DESCRIPTION OF THE INVENTION

One of the major challenges in treating CNS diseases is to identify the dose range of a drug resulting in the concentration in the brain sufficient to produce a therapeutic effect while avoiding unacceptable side effects. The present invention meets this challenge and provides effective methods for treating, preventing, or ameliorating a disease or condition by inhibiting NR2B subunit containing NMDA receptors by administering to a human in need of this treatment an effective amount of the compound of formula (I):

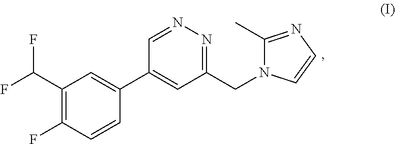

or a pharmaceutically acceptable salt thereof.

Preferably, the amount of the compound of formula (I) or its pharmaceutically acceptable salt that is administered for the treatment is from about 2 mg to about 50 mg per day. The total daily dose may be administered as single or divided doses. Such daily treatment amount or total daily dose may be from about 5 mg to about 45 mg, from about 6 mg to about 35 mg, from about 8 mg to about 30 mg, from about 10 mg to about 25 mg, from about 12 mg to about 20 mg, from about 14 mg to about 18 mg, from about 15 mg to about 18 mg, or any range among all of the above-listed amounts. For example, the daily treatment amount is from about 2 mg, 5 mg, about 6 mg, about 8 mg, or about 10 mg to about 12 mg, about 14 mg, about 15 mg, about 16 mg, about 18 mg, about 20 mg, about 25 mg, about 30 mg, or about 35 mg. In particular, the daily treatment amount is from about 2 mg, or about 4 mg to about 20 mg, about 25 mg, or about 30 mg.

Glutamate is the main excitatory neurotransmitter in the mammalian central nervous system (CNS) and mediates neurotransmission across most excitatory synapses. Three classes of glutamate-gated ion channels, alpha-amino-3-hydroxy-5-methyl-4-isoxazolepropionic acid (AMPA), kainate and N-methyl-D-aspartate (NMDA) receptors, transduce the post-synaptic signal. NMDA receptors are abundant, ubiquitously distributed throughout the brain, fundamental to excitatory neurotransmission and critical for normal CNS function. Two types of NMDA receptor subunits exist, NR1 and NR2(A-D), which combine to form functional NMDA receptors with different characteristics dependent upon the type of NR2 subunit they contain. The different NR2 subunits display different regional distribution in the CNS.

Historically, the clinical development of non-selective NMDA antagonists has suffered by a low therapeutic window due to mechanism-related CNS side effects. NR2B subtype selective NMDA antagonists, however, are potentially more advantageous.

Compounds selectively targeting the NR2B subunit containing receptors are generally known. For example, U.S. Pat. No. 7,005,432 discloses a broad variety of substituted imidazol-pyridazine derivatives that are NMDA receptor subtype selective blockers, which are said to be useful in the therapy of CNS disorders. It discloses that the dosage can vary within wide limits. In the case of oral administration the dosage is in the range of about 0.1 mg per dosage to about 1000 mg per day of a compound of general formula I of U.S. Pat. No. 7,005,432, although the upper limit can also be exceeded when this is shown to be indicated. Due to various factors, such as gastro-intestinal absorption, plasma-protein binding, and the ability of compounds to pass the blood-brain-barrier, it is not possible to predict in what amount a specific imidazol-pyridazine derivative would be effective.

The compound of formula (I) in accordance with the present invention is an NMDA NR2B subtype selective antagonist. NMDA receptors containing the NR2B subunit have been implicated in modulating functions, such as learning, memory processing, attention, emotion, mood, and pain perception, as well as being involved in a number of human disorders. Such disorders include, for example, cognitive impairment, neurodegenerative disorders, such as Alzheimer's disease and Parkinson's disease, pain (e.g. chronic or acute pain; neuropathic pain; post-operative pain), depression, attention deficit hyperactivity disorder, and addiction.

According to the present invention, an appropriate daily dose range of the compound of formula (I) or its pharmaceutically acceptable salt resulting in the concentration in brain sufficient to produce therapeutic effect while avoiding unacceptable side effects has been identified. The compound selectively modulates at such dose range the function of brain areas known as retrieval network, which is of importance in encoding and retrieval of memory. Therefore, it has applicability in the treatment of Alzheimer's disease. Furthermore, a selective increase in perfusion of the anterior cingulate cortex without a global effect on brain perfusion has been shown by the present invention. The anterior cingulate is a key functional junction of the brain with roles in monitoring behavior and adapting to feedback or conflict [Duncan and Owen (2000) Common regions of the human frontal lobe recruited by diverse cognitive demands. Trends Neurosci. 23: 475-83; Ridderinkhof et al. (2004) The role of the medial frontal cortex in cognitive control. Science 306: 443-7]. The areas of and around cingulate cortex are also important in pain responses, mood, and emotion. [Vogt (2005) Pain and emotion interactions in subregions of the cingulated gyrus. Nat Rev Neurosci 6:533-44]. Recent studies in mice indicate a central role for NMDA NR2B subunits in anterior cingulate mediated pain responses [Wei et al. (2001) Genetic enhancement of inflammatory pain by forebrain NR2B overexpression. Nat. Neurosci. 4: 164-9; Wu et al. (2005) Upregulation of forebrain NMDA NR2B receptors contributes to behavioral sensitization after inflammation. J. Neurosci 25: 11107-16]. Indeed, NR2B subunit containing NMDA receptors are thought to be critical for long term potentiation in the cingulate cortex and may therefore have a more general role in contextual emotion memory [Zhao et al. (2005) Roles of NMDA NR2B subtype receptor in prefrontal long-term potentiation and contextual fear memory. Neuron 47: 859-72]. Such anterior cingulate is rich in NR2B subunit containing NMDA receptors. Therefore, the compound of formula (I) and pharmaceutically acceptable salts thereof have applicability in the treatment of pain and depression.

### Example 1

A double-blind, placebo-controlled, randomized, single, and multiple oral dose study was conducted in order to determine safety and tolerability of the bis HCl salt of compound of formula (I) in healthy young and elderly subjects. The study was conducted in two parts.

Part 1 comprised an ascending single dose, sequential group study in 48 young male subjects, incorporating a two-period crossover arm to investigate the effect of food. Part 2 comprised an ascending multiple dose, sequential group study in 24 young male subjects and an ascending single and multiple dose, sequential group study in 18 elderly subjects (10 males and 8 females) subjects. On each dosing occasion, the subjects received a single capsule containing the appropriate amount of either the bis HCl salt of compound of formula (I) or microcrystalline cellulose. Treatments were administered orally with 240 ml water while the subjects were in a standing position.

The condition of each subject was monitored throughout the study. In addition, any signs and symptoms were observed and elicited at least once a day by open questioning, such as “How have you been feeling since you were last asked?” Subjects were also encouraged to report spontaneously any adverse events during the study.

Any adverse events or remedial actions were recorded. The nature, time of onset, duration, and severity were documented. Any clinically significant abnormalities found during the course of the study were followed up until they returned to normal or could be clinically explained.

A serious adverse event is defined as any untoward medical occurrence that at any dose either results in death, is life-threatening, requires inpatient hospitalization, or prolongs existing hospitalization, results in persistent or significant disability/incapacity, and/or results in a congenital anomaly/birth defect.

Important medical events that may not result in death, be life-threatening, or require hospitalization may be considered to be serious adverse events when, based upon appropriate medical judgment, they may jeopardize the subject or may require medical or surgical intervention to prevent one of the above-described outcomes.

The results of the study showed that the bis HCl salt of compound of formula (I) was very well tolerated when administered to young males as single oral doses of up to 15 mg (including single oral doses of 2, 5, and 10 mg), and multiple oral doses (over 8 days) of up to 8 mg once daily. Similarly, the bis HCl salt of compound of formula (I) was found to be very well tolerated when administered to elderly males and females as single oral doses of up to 4 mg, and multiple oral doses (over 8 days) of up to 3 mg twice daily. A low incidence of mainly mild adverse events was reported throughout the study, and there were no serious or severe adverse events and no subject was withdrawn as a result of adverse events. There were no apparent trends in the vital signs and electrocardiogram (ECG) parameters following single or multiple dosing in young or elderly subjects.

### Example 2

A double blind, placebo controlled study was conducted in 19 healthy volunteers to investigate the role of NMDA receptor NR2B subunit selective antagonism on cognitive functions and neurophysiology as measured with magnetic resonance imaging (MRI). This study utilized functional MRI to determine changes in regional cerebral blood flow (rCBF) and the putative modulation of neural networks during performance of cognitive tasks by the subjects after they were administered the bis HCl salt of compound of formula (I).

### Experimental Procedure

The 19 healthy male volunteers who participated in this study were scanned using functional magnetic resonance imaging (fMRI) whilst performing cognitive tasks (including the PAL task described below in more detail) designed to measure associative learning, sustained attention, and episodic memory. These tasks were repeated on three study days two hours prior to the fMRI session. On these three separate occasions, the participants received a capsule containing either placebo (microcrystalline cellulose), 8 mg, or 15 mg of the bis HCl salt of compound of formula (I) such that all treatments were given to all participants. During the fMRI session, the tasks were presented in a randomized order to each subject; however, the order assigned was then maintained across all three study sessions. Additional practice on the tasks was offered to the participants before entering the scanner, and the instructions were repeated prior to each task being performed whilst in the scanner.

### Paired Associate Learning (PAL)

This task required learning of stimulus-location associations. Initially, six distinct patterns appeared on the screen, one by one in a pseudorandom order. Each pattern appeared in a different location and remained there for one second. After the last pattern was revealed, the six patterns were then shown one by one in the centre of the screen for four seconds. Participants responded to each stimulus by moving the joystick towards the position they believed to be the original location. This cycle was presented twice more with the same stimuli in the same locations, but shown in a different order. Thus, in one block of the task, participants had three opportunities (named A, B and C) to learn the locations of the 6 stimuli. Overall, the task consisted of six blocks of stimuli, each time with a new set of patterns. The control condition involved viewing a single stimulus appearing in each location followed by the same stimulus appearing in the centre of the screen accompanied by a grey circle highlighting the direction in which the joystick should be moved. The control condition was also presented six times, controlling for the visual and motor requirements within the same framework as the learning conditions. The total task length was 12 minutes and 12 seconds.

### Imaging Procedure

Images were acquired on a 1.5T GE Excite HDx system (General Electric, Milwaukee, Wis.). For all tasks a gradient-echo EPI sequence was used with TE=40 ms, FOV=24 cm, and in-plane resolution of 3.75 mm² (matrix=64²). Thirty-eight axial slices with thickness 3 mm (0.3 mm gap) approximately parallel to the AC/PC line were obtained. For the paired associates learning task TR=3000 ms. In addition, a high-resolution image was acquired using single shot EPI with TR=3000 ms, TE=40 ms, FOV=24 cm, 3 mm slice thickness with 0.3 mm gap, matrix=128², and 43 slices. This image was used to determine coregistration parameters.

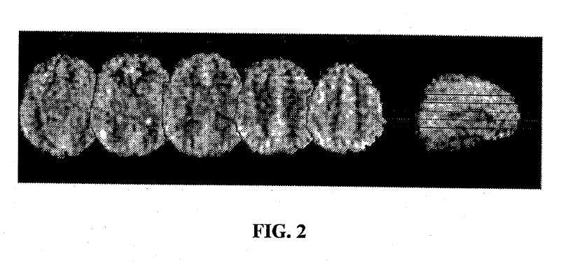

Whole-brain resting perfusion images were acquired with a continuous arterial spin labeling (CASL) sequence consisting of a pseudo-continuous flow-driven, adiabatic inversion scheme and a continuous 3D fast spin echo (FSE) sequence with an interleaved stack of variable density spirals acquisition (Alsop and Detre (1998) Multisection cerebral blood flow MR imaging with continuous arterial spin labeling. Radiology 208: 410-6). An example of images acquired using this technique is shown in FIG. 2. CASL perfusion images are able to delineate grey and white matter and provide high resolution quantification of rCBF across the whole brain. Because of the genuine 3-dimensional encoding and readout of this multi-shot technique, and the refocusing of magnetic susceptibility induced signal distortions, the maps of whole brain rCBF obtainable with this technique are of good image quality and exquisite spatial resolution.

### Preprocessing

For the perfusion images, brain volumes were first normalized into a standard anatomical space (International Consortium on Brain Mapping—ICBM), using affine registration and non-linear transformations. Normalization utilized a single image to which all three CASL sessions were coregistered in order to reduce the likelihood of preprocessing-dependent differences between the drug and placebo session images. All images were spatially smoothed using a Gaussian kernel full-width at half-maximum filter of 6×6×6 mm to improve signal-to-noise ratio and allow for inherent functional and gyral variability across participants.

### MRI Data Analysis

Image preprocessing and analysis were carried out using Statistical Parametric Mapping software developed by the Functional Imaging Laboratory, UCL, (SPM v5.0, www.fillon.ucl.ac.ulc/spm/)., running under Matlab 7.0.1, and a blood oxygen level dependency (BOLD) was determined. BOLD changes to cognitive tasks represent changes of neuronal activities of respective brain areas. Entire brain volume was analyzed using the general linear model, with statistical thresholding restricted on the basis of the general regional hypothesis of brain areas of particularly high NR2B receptor subtype density and relevant for cognitive enhancement of mnemonic function. Absolute thresholding of 15 ml/min/100 ml was used to minimize the contribution of white matter regions to the analysis. However, removal of this thresholding did not alter the pattern of findings observed. Appropriately weighted linear contrasts were used to produce statistical maps of the following:

1. Areas of increased/decreased perfusion following administration of the bis HCl salt of compound of formula (I) (averaged over 8 mg and 15 mg): Main effect of drug.

2. Areas of increased/decreased perfusion following administration of 8 mg of the bis HCl salt of compound of formula (I).

3. Areas of increased/decreased perfusion following administration of 15 mg of the bis HCl salt of compound of formula (I).

4. A dose response relationship between the bis HCl salt of compound of formula (I) at 8 mg and 15 mg, ignoring the effect of placebo (orthogonal to comparison 1)

### Neuroimaging Analysis of Regional Cerebral Blood Flow

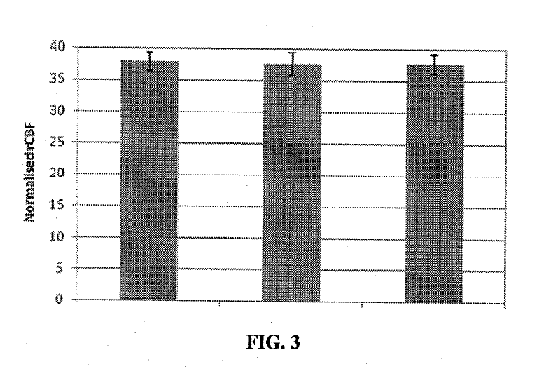

Global perfusion was first calculated using the continuous arterial spin labeled perfusion maps, normalized to a standard space (ICBM) and skull-stripped using the brain mask provided with Statistical Parametric Mapping software (SPM v5.0). A lower cut-off for white matter was not used. The analysis of the global perfusion values showed no difference across the three conditions studied [F (2,34)=2.62, P=0.77]; see FIG. 3.

The regional effects of the administered bis HCl salt of compound of formula (I) on absolute perfusion were analyzed using SPM v5.0. At a threshold of p=0.05 after correction for multiple comparisons across the whole brain, there were no significant clusters where signal differed across the three conditions studied.

Normalization of local rCBF changes to the global signal allowed examination of local changes relative to global signal and proved to be a more sensitive analysis technique. Using the normalized maps of rCBF, increased signal following administration of the his HCl salt of compound of formula (I) was seen in a discrete cluster at the genu of the anterior cingulate cortex, extending ventrally (x,y,z=8, 42, 10, BA25, T=5.41, p(corr)=0.023). Coordinates are given according the system of Talairach and Tourneux (Co-planar stereotaxic atlas of the human brain. Thieme, Stuttgart, 1988) in relation to the standard image space of the ICBM (MNI152). Brodmann areas refer to the cytoarchitectonically defined brain regions.

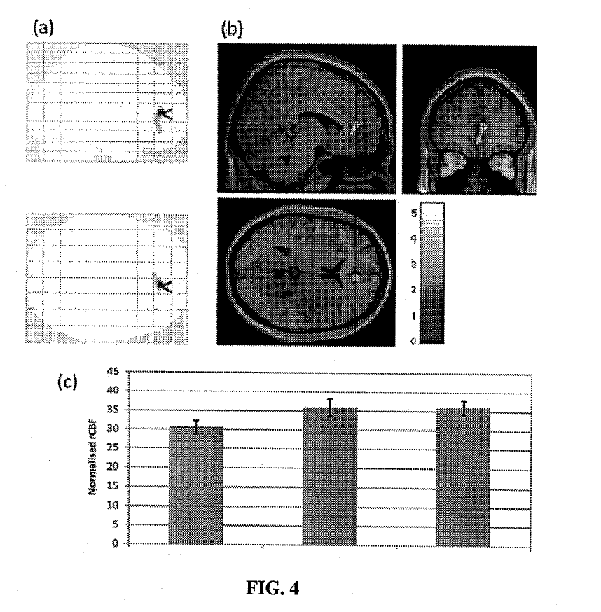

The increases in rCBF were 17.5% for 8 mg and 17.9% for 15 mg of the bis HCl salt of compound of formula (I). The two doses did not differ for this region. FIG. 4 shows the location of the change in perfusion and illustrates the size of the effect of the bis HCl salt of compound of formula (I).

Although the effect size for the cluster of increased rCBF shown in FIG. 4 was similar for both doses of the bis HCl salt of compound of formula (I), separate analyses of each dose suggested that the effect was driven by the higher dose. Direct comparison of the bis HCl salt of compound of formula (I) at 15 mg with placebo revealed a highly significant cluster of increased rCBF in the same region as the contrast of both doses against placebo (x,y,z=8, 42, 10, BA25, T=5.74, p(corr)=0.037). There were no clusters of voxels showing significant decreases in rCBF with the his HCl salt of compound of formula (I).

In relation to the contrasts specified in the MRI data analysis section the results are summarized below:

1. Areas of increased/decreased perfusion following administration of the bis HCl salt of compound of formula (I) (averaged over 8 mg and 15 mg): Main effect of drug. An increase in perfusion was seen in the anterior cingulate (peri-genual).

2. Areas of increased/decreased perfusion following administration of 8 mg of the bis HCl salt of compound of formula (I). No statistically significant changes were seen presumably due to low sample size.

3. Areas of increased/decreased perfusion following administration of 15 mg of the bis HCl salt of compound of formula (I). An increase in perfusion was seen in the anterior cingulate (peri-genual). This is an area rich in NMDA receptors, which has been implicated as significant for indications of the central nervous system, such as AD, neuropathic pain, depression, Parkinson's disease, and the other indications mentioned above.

4. A dose response relationship between the bis HCl salt of compound of formula (I) at 8 mg and 15 mg, ignoring the effect of placebo (orthogonal to comparison 1). No changes were seen in the formal comparison across the entire brain volume.

The specific region of the anterior cingulate cortex modulated by the administered bis HCl salt of compound of formula (I) at the genu, extending inferiorly, shows strong connectivity with other forebrain regions, including the medial wall of the prefrontal cortex, the ventral portion of the striatum, thalamus, and posterior superior parietal lobule [Margulies et al. (2007) Mapping the functional connectivity of anterior cingulate cortex. Neuroimage 37: 579-88]. Thus, tasks that recruit these connected regions may be sensitive to the effects of the compound of formula (I) and salts thereof, including tasks where the core process of valuation of a reward is important. This does not discount an effect on mnemonic function as highlighted by research in experimental animals [Higgins et al. (2005) Evidence for improved performance in cognitive tasks following selective NR2B NMDA receptor antagonist pre-treatment in the rat. Psychopharmacology (Berl) 179: 85-98], but shows a broader role for NR2B receptor modulation.

### Neuroimaging Analysis of Cognitive Task Networks

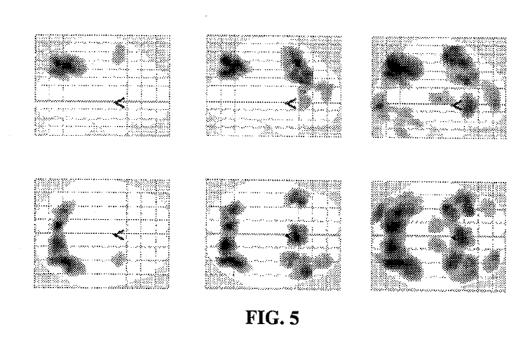

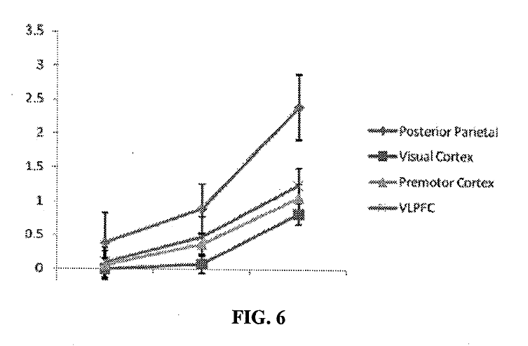

While the functional performance of subjects in PAL was unaffected by administration of the bis HCl salt of compound of formula (I), as expected for healthy young subjects who frequently perform at an optimum level, the analysis of the BOLD response revealed surprising findings: the activity of a number of brain regions, known for their role as a memory retrieval network (e.g., posterior parietal region, visual cortex, premotor cortex and ventrolateral prefrontal cortex [VLPFC]), were selectively increased during the performance of certain tasks. Preliminary analysis of the effects of the administered bis HCl salt of compound of formula (I) suggest dose dependent increases in BOLD signal during retrieval in key cortical nodes of activation, with additional dose dependency in subcortical regions. FIG. 5 shows glass brain maximum intensity projections showing the statistically significant clusters of activation during retrieval in the Paired Associates Learning task (PAL) after administration of placebo (left: upper and lower panel), 8 mg of bis HCl salt of compound of formula (I) (middle: upper and lower panel), and 15 mg of his HCl salt of compound of formula (I) (right: upper and lower panel), respectively. Upper images are from the left and lower images are from above. Furthermore, FIG. 6 shows concentration response curves for the activation contrast in respective brain regions as indicated. Points on the left, middle, and right correspond to placebo, 8 mg, and 15 mg of bis HCl salt of compound of formula (I), respectively. Data are extracted from activation patterns during retrieval in the Paired Associates Learning task (PAL).

The dose-dependent increase, by the bis HCl salt of compound of formula (I), in activity of the memory retrieval network during the performance of this cognitive task indicates a specific pharmacological effect relevant for the treatment of Alzheimer's disease and cognitive impairment.

### Example 3

The use of CSF sampling in drug development, to assess drug or disease response, has increasingly demonstrated relevance. The value of CSF sampling for the assessment of central drug penetration and measurement of bioactive substances has long been acknowledged. Generally, CSF biomarkers enable therapeutic candidates to be examined more effectively, exposing fewer people to ineffective drugs and doses, and speeding the identification of effective therapies. Lumbar punctures (LPs) are performed routinely in a variety of clinical settings for diagnosis and therapy (as in intra-thecal delivery of anaesthetics, analgesics and chemotherapeutics) (Roos (2003) Lumbar puncture. Semina Neurol. 23(1): 105-14). The cerebrospinal fluid is produced by the ventricles (mostly the lateral ventricles) at a rate of 500 ml/day. Since the volume that may be contained by the brain is of the order of 150 ml, it is frequently replaced (3-4 times per day turnover), exceeding amounts getting into the blood. This continuous flow through the ventricular system into the subarachnoid space and finally exiting into the venous system provides somewhat of a “sink” that reduces the concentration of larger, lipoinsoluble molecules penetrating into the brain and CSF (Saunders et al. (1999) Barrier mechanisms in the brain, II. Immature brain. Clin Exp Pharmacol Physiol. 26(2): 85-91).

One lumbar puncture per subject was performed on day 8 of treatment (daily dose of 8 mg of bis HCl salt of the compound of formula (I)), in the timeframe of 3 to 4 hours after dosing.

All care was taken to avoid contamination of the collected CSF sample with blood. Thus, only clear CSF was collected, after blood-contaminated liquid has left the cannula. Bloody taps were discarded.

Samples were aliquoted per time point for the measurement of compound of formula (I) as follows: a 0.5 ml CSF sample was aliquoted in empty dry polypropylene tubes from the ≈5 ml CSF collected at each scheduled time point.

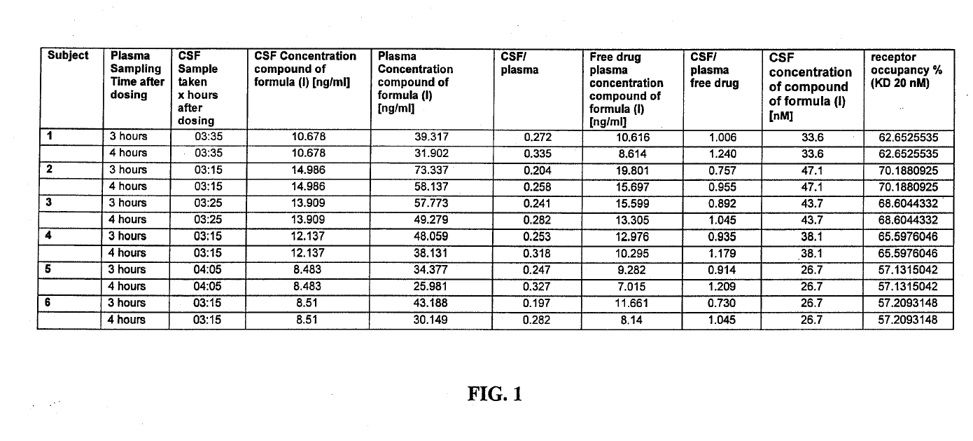

CSF samples were stored immediately in an upright position at a temperature of −70° C. or below until shipment. Compound of formula (I) concentrations were measured by a specific and validated LC-MS-MS method. Cerebrospinal fluid (CSF) penetration has been assessed in six healthy subjects receiving 8 mg of bis HCl salt of compound of formula (I) daily for 8 days. As shown in FIG. 1, CSF concentration of compound of formula (I) largely corresponds to the free (unbound) plasma concentrations of such compound. Such concentrations in the CSF have been extrapolated to estimate the extent of occupancy of the NR2B subtype of the NMDA receptor. It was found that at such daily dose of 8 mg of compound of formula (I) receptor occupancy is greater than that, which corresponds to currently used therapeutic dosages of memantine in Alzheimer's disease. The selectivity of the compound of formula (I) and salts thereof in combination with the good penetration of the blood brain barrier at well-tolerated dosages according to the present invention provides a therapeutic advantage.

1. A method of treating, preventing or ameliorating a disease or condition by inhibiting NR2B subunit containing NMDA receptors comprising administering to a person in need of such treatment a total daily dose from about 2 to about 50 mg of a compound of formula (I): 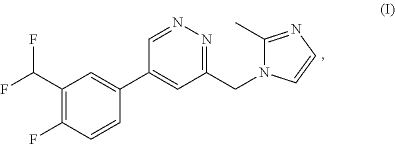
or a pharmaceutically acceptable salt thereof.

2. The method according to claim 1, wherein the disease or condition is cognitive impairment, a neurodegenerative disease, pain, depression, attention deficit hyperactivity disorder, or addiction.

3. The method according to claim 2, wherein the neurodegenerative disease is Alzheimer's disease or Parkinson's disease.

4. The method according to claim 2, wherein the pain is chronic or acute pain.

5. The method according to claim 2, wherein the pain is neuropathic pain or post-operative pain.

6-11. (canceled)

12. The method according to claim 4, wherein the pain is neuropathic pain or post-operative pain.
